(19) United States
(12) Patent Application Publication
(10) Pub. No.: US 2025/0001151 A1
(43) Pub. Date: Jan. 2, 2025

(54) HALOTHERAPY MODULE NOZZLE

(71) Applicant: Sauna Works Inc. (aka Far Infrared Sauna Technology Co.), Berkeley, CA (US)

(72) Inventors: Raleigh C Duncan, Berkeley, CA (US); Andrew Kaps, San Francisco, CA (US)

(73) Assignee: Sauna Works Inc. (aka Far Infrared Sauna Technology Co.), Berkeley, CA (US)

(21) Appl. No.: 18/886,968

(22) Filed: Sep. 16, 2024

Related U.S. Application Data
(63) Continuation-in-part of application No. 18/155,685, filed on Jan. 17, 2023.
Application No. 18/155,685 is a continuation of application No. 17/662,177, filed on May 5, 2022.
(60) Provisional application No. 63/185,261, filed on May 6, 2021.

(51) Int. Cl. A61M 35/00; A61H 33/06; A61M 11/00; G16H 20/90

(52) U.S. Cl. CPC A61M 35/20; A61H 33/063; A61M 11/005; A61H 2201/0107; A61H 2201/0207; A61H 2201/105; A61H 2201/5007; A61H 2201/5058; G16H 20/90

(57) ABSTRACT

Systems include a receiving port configured to receive a cartridge configured to store a material capable of being aerosolized, a sensor configured to generate one or more measurements based on ambient conditions of a housing, an aerosolizer configured to aerosolize the material in response to receiving a signal, a user replaceable aerosolizer nozzle, and a controller comprising one or more processors configured to generate the signal provided to the aerosolizer, and further configured to control operation of the aerosolizer via the signal.

### CROSS-REFERENCE TO RELATED APPLICATIONS

This application is a continuation-in-part of U.S. patent application Ser. No. 18/155,685, filed Jan. 17, 2024, which is a continuation of U.S. patent application Ser. No. 17/662,177, filed May 5, 2022, which claims the benefit under 35 U.S.C. § 119 (e) of U.S. Provisional Patent Application No. 63/185,261, filed on May 6, 2021, which are all incorporated herein by reference in its entireties for all purposes.

### TECHNICAL FIELD

The present disclosure relates generally to saunas, and more specifically to halotherapy modules capable of being implemented in saunas.

### DESCRIPTION OF RELATED ART

Saunas may be rooms or enclosed areas in which heat may be used to make users of the saunas perspire. Accordingly, an enclosed area of a sauna may be heated such that the temperature inside the sauna is elevated relative to an exterior temperature. Accordingly, such saunas are able to generate and retain heat, and such heat may be used for therapeutic purposes. While saunas are capable of generating heat for therapeutic purposes, they remain limited in their ability to do so in combination with other therapeutic modalities.

### BRIEF DESCRIPTION OF THE DRAWINGS

The disclosure may best be understood by reference to the following description taken in conjunction with the accompanying drawings, which illustrate particular embodiments.

FIG. 1 illustrates an example of a halotherapy module, configured in accordance with some embodiments.

FIG. 2 illustrates an example of a sauna, configured in accordance with some embodiments.

FIG. 3 illustrates using a halotherapy module, implemented in accordance with some embodiments.

FIG. 4 illustrates using a halotherapy module, implemented in accordance with some embodiments.

FIG. 5 illustrates a halotherapy module, implemented in accordance with some embodiments.

FIG. 6 illustrates halotherapy nozzle clog detection.

FIG. 7 illustrates an example of a front side of a halotherapy device.

FIG. 8 illustrates an example of a back side of a halotherapy device.

FIG. 9 illustrates one example of a controller, configured in accordance with some embodiments.

### DESCRIPTION OF EXAMPLE EMBODIMENTS

Reference will now be made in detail to some specific examples including the best modes contemplated by the inventors. Examples of these specific embodiments are illustrated in the accompanying drawings. While various embodiments are disclosed herein, it will be understood that they are not intended to be limiting. On the contrary, they are intended to cover alternatives, modifications, and equivalents as may be included within the spirit and scope of the disclosure as defined by the appended claims.

For example, the techniques will be described in the context of saunas, and heating elements associated with such saunas. However, it should be noted that the techniques apply to a wide variety of different environments and enclosures. In the following description, numerous specific details are set forth in order to provide a thorough understanding of the present invention. Particular example embodiments may be implemented without some or all of these specific details. In other instances, well known process operations have not been described in detail in order not to unnecessarily obscure the present disclosure.

Various techniques and mechanisms of the present disclosure will sometimes be described in singular form for clarity. However, it should be noted that some embodiments include multiple iterations of a technique or multiple instantiations of a mechanism unless noted otherwise. For example, a system uses a processor in a variety of contexts. However, it will be appreciated that a system can use multiple processors while remaining within the scope of the present disclosure unless otherwise noted. Furthermore, the techniques and mechanisms of the present disclosure will sometimes describe a connection between two entities. It should be noted that a connection between two entities does not necessarily mean a direct, unimpeded connection, as a variety of other entities may reside between the two entities. For example, a processor may be connected to memory, but it will be appreciated that a variety of components may reside between the processor and memory. Consequently, a connection does not necessarily mean a direct, unimpeded connection unless otherwise noted.

### Overview

Techniques and mechanisms described herein provide for the configuration and utilization of halotherapy modules. As will be discussed in greater detail below, such halotherapy modules may be implemented as stand-alone devices, or may be implemented and/or integrated in the context of a sauna. In various embodiments, such halotherapy devices may utilize vibrational meshes or nebulizing meshes to aerosolize material for the implementation of one or more therapies for a user, such as a halotherapy. In some examples, the halotherapy devices include user replacement aerosolizer nozzles. As will also be discussed in greater detail below, such halotherapy modules may be configured to operate in conjunction with other components of a sauna, such as heating elements, to provide comprehensive therapies for the user.

### EXAMPLE EMBODIMENTS

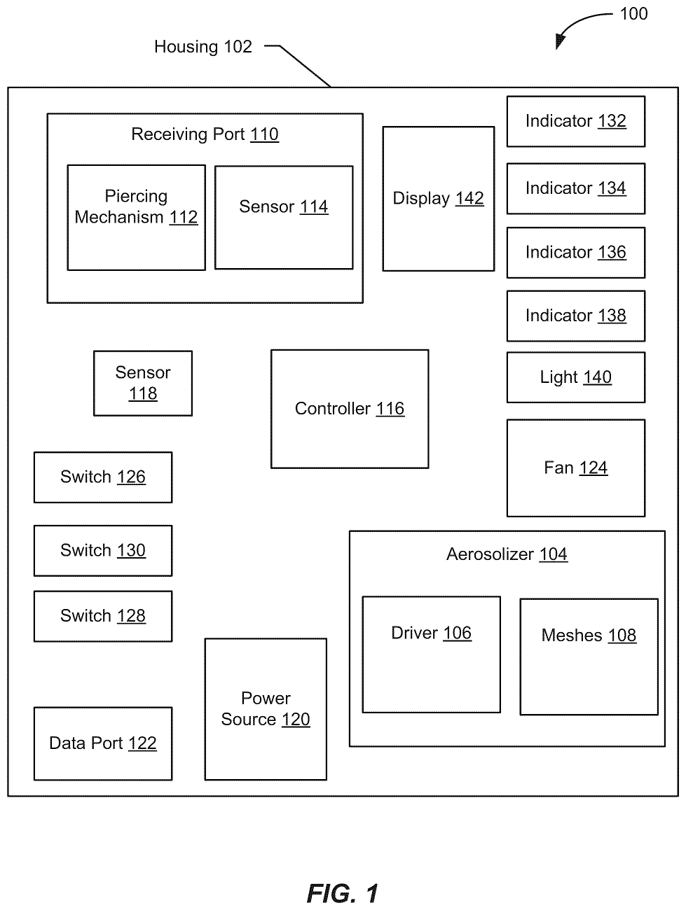

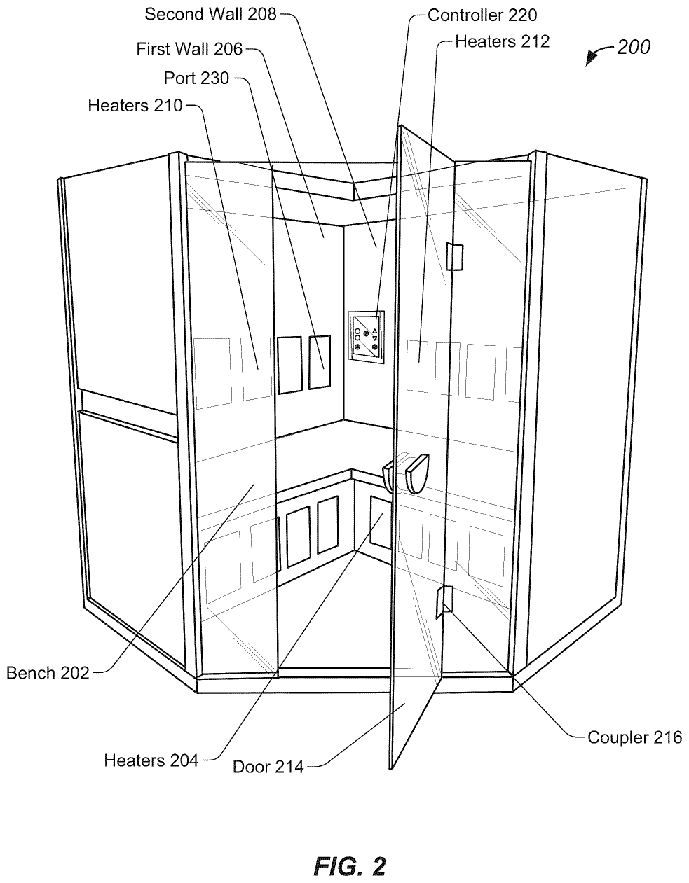

FIG. 1 illustrates an example of a halotherapy module, configured in accordance with some embodiments. As will be discussed in greater detail below, a halotherapy module, such as halotherapy module 100, may be configured to aerosolize one or more materials in a configurable manner, and provide such aerosolized material to an environment external to the module. In various embodiments, the material may be a material that includes salt, and may be used for the purposes of halotherapy in the context of a sauna. Additional details regarding a sauna are discussed in greater detail below with reference to FIG. 2. Accordingly, halotherapy module 100 may be configured to aerosolize multiple different materials for various therapeutic treatments implemented in a sauna.

In various embodiments, halotherapy module 100 includes housing 102 which is configured to house components of halotherapy module 100. As will be discussed in greater detail below, housing 102 may be further configured to be removably coupled with a housing of a sauna. In this way, housing 102 may be configured to enable mechanical coupling and decoupling with a sauna to enable halotherapy module 100 to be used in the context of a sauna, or as a stand-alone device for use in another context, such as a room or office. As will also be discussed in greater detail below, the coupling between halotherapy module 100 and the sauna may be via a wall mount, a recessed portion, or any other suitable coupling mechanism.

As shown in FIG. 1, various components of halotherapy module 100 are included in housing 102. For example, aerosolizer 104 may be included in housing 102. As shown in FIG. 1, aerosolizer 104 may include various components such as driver 106 and meshes 108 that are configured to aerosolize one or more materials via mechanical vibration. Accordingly, driver 106 may be a mechanical driver that is configured to vibrate one or more of meshes 108 at a designated frequency. Moreover, meshes 108 may be metal or wire meshes that are configured to have specific dimensions of permeability.

For example, a size of holes or gaps in a mesh layer may determine a size of aerosolized particles that are generated. In one example, meshes 108 may be configured to have permeability features, such as fine holes with a circular geometry, configured to generate particle sizes of a range of 1-10 microns. In various embodiments, meshes 108 may include multiple mesh layers which may have the same or different dimensions and geometries. In some embodiments, driver 106 is configured to drive all mesh layers simultaneously, or may be configured to drive each layer independently. When driven, a particular mesh layer may vibrate and move back and forth in a designated axis of movement. When a material contacts such a vibrating mesh and passes through holes when vibrating, it is broken down into small, consistent particles that may be suspended in the air, and thus aerosolized.

According to various embodiments, a meshes 108 are nebulizing meshes having features tuned for effective nebulizing of saline solution. In specific embodiments, a nebulizing mesh has a diameter of 20 mm±0.5 mm, a height of 3 mm±0.3 mm, a resonance frequency of 108.0±6.0 kHz, a resonance impedance of <=260.0 Ohms. According to various embodiments, a micro-pole diameter is 5 μm and the manufacturing process involves laser drilling through stainless steel.

In particular embodiments, a nebulizing mesh in meshes 108 work with a saline cartridge with a controlled volume of 25 ml±1 ml. A feedback loop included on a circuit board can include a feedback loop to maintain a resonance frequency of the mesh for a constant nebulizing process. In some embodiments, the nebulizing rate is controlled based on the amount of water inside the water tank of the unit. A low water level detector such as an optical sensor can pause the unit when the level is too low, which could create risk to the nebulizing process.

According to various embodiments, a two speed fan system or a multiple speed fan system is placed to further enhance coverage of therapy, for coverage up to an area of 30-40 square meters.

In various embodiments, housing 102 further includes a receiving port, such as receiving port 110, that is configured to receive a cartridge of a material. In one example, receiving port 110 is configured to receive a disposable cartridge or capsule that includes a material to be aerosolized. For example, the material may be a saline solution, and the saline solution may be aerosolized by aerosolizer 104 discussed above. In particular embodiments, the saline cartridge has a 3% salt concentration. In some embodiments, the saline cartridge can be effective with a 2%-%5 salt concentration.

Various embodiments of the present invention recognize the particular effectiveness of a 3% saline solution, more specifically a 3.342% saline solution.

For example, if

$$a = required dry salt output rate of \text{.8} g / 20 m$$

$$b = saline solution concentration$$

$$c = required saline solution output rate = a / b = \text{.8} / b [ g / hr ]$$

$$d = saline solution density = \text{.0075} \times b + \text{.9972} [ g / cc ]$$

$$e = unit conversion of saline solution output rate = c / 20 / d [ cc / min ]$$

$$f = required mesh amount = 2 [ pcs ]$$

$$g = cable salilne solution outpu rate = f \times 0.6 cc / min = 2 \times 6 = 1.2 [ cc / min ]$$

$$c / 20 / d = 1.2$$

$$\frac{\text{.8}}{b} * \frac{1}{20} * \frac{1}{\text{.0075} * b = \text{.9972}} = 1.2$$

$$\text{.0075} {b}^{2} + \text{.9972} b - \frac{\text{.08}}{1.2 * 20} = 0$$

$$b = 0.3342 = 3.342 \%$$

Various embodiments of the present invention also recognize particular benefits of optimizing salt particle diameter, but also recognize that preferred salt particle diameters may vary depending on preferences and therapy required. In some examples, a saline solution particle diameter of 2 μm is used to result in a dry salt particle diameter of 0.5020 μm.

For example,

$$a = saline solution particle diameter [ μ m ]$$

$$b = saline solution particle volume = \frac{4}{3} * π * {( \frac{a * {10}^{- 4}}{2} )}^{3} [ {m}^{3} ]$$

$$c = saline solution particle mass = b \times 1022.265 [ kg ]$$

$$d = salt mass in saline solution particle c * 3.342 \% [ kg ]$$

$$e = salt particle volume in saline solution particle = d / 2160 [ {m}^{3} ]$$

$$f = resultant dry salt particle diameter = \sqrt[3]{e * \frac{3}{4} * \frac{1}{π}} * {10}^{4} * 2 [ μ m ]$$

According to various embodiments, a saline solution particle diameter of 2 μm, 5 μm, and 12 μm, results in a dry salt particle diameter of 0.5020 μm, 1.2551 μm, and 3.0122 μm respectively.

Accordingly, receiving port 110 may be coupled to aerosolizer 104 such that a material received at receiving port 110 is passed through aerosolizer 104. In one example, tubing may couple receiving port 110 to aerosolizer 104, and receiving port 110 may include a pump configured to pump contents of the cartridge to aerosolizer 104.

As shown in FIG. 1, receiving port 110 may include one or more components configured to facilitate the intake of material from a cartridge. For example, receiving port 110 may include cartridge foil piercing mechanism 112, such as a hollow needle, and one or more sensors, such as sensor 114 which is configured to detect a presence of a cartridge, and provide such an indication to controller 116 discussed in greater detail below. In some embodiments, sensor 114 may identify one or more features, such as an identifier of the cartridge, and may provide such identifiers to controller 116, which may determine one or more operational parameters based on such identifier. For example, sensor 114 may detect an identifier that identifies one or more aspects of the contents of the cartridge, such as an indicator of concentration, or a viscosity of the contents. The identifier may be mapped to a particular set of operational parameters based on a predetermined mapping stored in memory of controller 116. Such a predetermined mapping may have been determined by an entity, such as a manufacturer. The identifier may be identified based on an optical scanner that may be included in sensor 114, or one or more mechanical sensors included in sensor 114. For example, mechanical protrusions, such as pins, may be present on an exterior of the cartridge, and may depress one or more mechanical sensors, such as buttons, when the cartridge is inserted. The pattern of buttons pressed may correspond to an identifier, and be mapped to operational parameters based on the mapping determined by the manufacturer. In this way, operation of aerosolizer 104 may be configured based on identifiers provided by the cartridge.

As discussed above, housing 102 additionally includes controller 116 which is configured to control operation of aerosolizer 104 as well as other components of halotherapy module 100, as will be discussed in greater detail below. Accordingly, controller 116 includes one or more processors and a memory configured to store instructions associated with the one or more processors, as well as various data values and parameters associated with the programmatic operation of the one or more processors. In this way, controller 116 may configure aspects of aerosolizer 104 in a configurable manner. For example, controller 116 may determine a vibrational frequency and intensity to be applied, and a sequence of activation for aerosolizer 104. As will also be discussed in greater detail below, controller 116 may be communicatively coupled to a controller of a sauna, and may thus control operation of aerosolizer 104 in coordination with other components of a sauna, such as heating elements. In this way, controller 116 may control aerosolizer 104 based, at least in part, on inputs received from a sauna as well as sensors included in the sauna. In this way, operation of aerosolizer 104 may be coordinated with other components of a sauna, such as heaters, as part of a comprehensive treatment program executed by one or more controllers.

In various embodiments, housing 102 includes one or more sensors, such as sensor 118, that are configured to monitor one or more conditions of halotherapy module 100, and provide an input to controller 116. In various embodiments, sensor 118 is a temperature sensor that is configured to provide one or more temperature readings to controller 116. The temperature reading may be a temperature of halotherapy module 100, as well as a temperature of contents of a cartridge coupled to receiving port 110. In various embodiments, controller 116 may use such temperature readings to generate operational parameters for aerosolizer 104. For example, controller 116 may determine a particular vibrational frequency and intensity for driver 106 and meshes 108 based on the temperature readings provided by sensor 118. In some embodiments, such a determination may be made based on a look up table stored in a memory of controller 116 that may have been stored during a configuration operation, as may have been implemented by a manufacturer.

In various embodiments, housing 102 additionally includes power source 120 which may include one or more sources of power for component of halotherapy module 100. For example, power source 120 may include a rechargeable battery, and may also include a charging port. In some embodiments, power source 120 is a high temperature battery configured to operate at temperatures around 140 degrees Fahrenheit. Moreover, power source 120 may include a port configured to be coupled to an electrical outlet, such as a wall socket. In this way, power source 120 may be a portable power supply, such as a rechargeable battery and/or may be a wired connection that couples to an external power supply, such as that of an electrical socket included in a wall, or a power supply of a sauna.

As discussed above, housing 102 further includes data port 122 which is configured to establish one or more data connections with external devices. As previously discussed, halotherapy module 100 may communicatively coupled to one or more processing devices of a sauna. Accordingly, such communicative coupling may be implemented via data port 122. In some embodiments, data port 122 may also enable communicative coupling with other devices, such as a user's mobile device or personal computer. In this way, halotherapy module 100 may be communicatively coupled with an application implemented on a mobile communications device, and may be controlled via such a mobile application. Moreover, it will be appreciated that data port 122 may be a wireless data connection, such as a Bluetooth or WiFi connection, thus enabling wireless communication with such other external devices.

Housing 102 further includes fan 124 which is configured to circulate air, and blow air in a designated direction. Accordingly, fan 124 may be configured to blow air past meshes 108, and thus expel aerosolized material via an exhaust port of housing 102. In this way, operation of fan 124 may facilitate distribution of aerosolized material in an operational environment of halotherapy module 100, such as a sauna. In various embodiments, fan 124 is controlled by controller 116. Accordingly, controller 116 may determine an activation and deactivation sequence for fan 124, as well as a rotational frequency for fan 124.

Housing 102 additionally includes various switches, such as switch 126, switch 128, and switch 130. In various embodiments, the switches may be tactile switches that are manipulated by a user, and are configured to provide input signals to one or more components of halotherapy module 100, such as controller 116. For example, switch 126 may be a power/standby switch that is operable by a user. Such a switch may be used by a user to manually turn on and off halotherapy module 100. Moreover, switch 128 may be a light control switch that a user may use to manually control a light integrated into housing 102, such as light 140. Additionally, switch 130 may be a fan control switch that a user may use to manually control fan 124.

Housing 102 further includes various indicators such as indicator 132, indicator 134, indicator 136, and indicator 138. In various embodiments, the indicators may be dedicated visual indicators configured to provide specific information to a user. For example, indicator 132 may be an error indicator that provides a user with a visual indicator if any error condition is detected by halotherapy module 100. Moreover, indicator 134 may provide a visual indicator of a power and battery status. Furthermore, indicator 136 may provide a visual indication of operation of fan 124, such as an indication of a fan speed. Further still, indicator 138 may provide an indication of an operation of light 140, such as on/off status as well as an intensity setting.

Housing 102 additionally includes a display, such as display 142 which may be configured to display various information to a user Accordingly, display 142 may be a numeric light emitting diode (LED) display that may display various information, such as a current fan and aerosolizer setting. In some embodiments, display 142 may be a display panel, such as a liquid crystal display. Accordingly, display 142 may be configured to provide a user interface to the user, and a comprehensive display of operational parameters of halotherapy module 100.

FIG. 2 illustrates an example of a sauna, configured in accordance with some embodiments. As will be discussed in greater detail below, sauna 200 may be a climate and humidity-controlled enclosure which includes various heating elements configured to provide heat to one or more users that may be included inside the enclosure of sauna 200. As will also be discussed in greater detail below, sauna 200 is configured to include a halotherapy module, such as halotherapy module 100 discussed above.

Thus, according to various embodiments, sauna 200 may be an enclosure that is configured to accommodate one or more users in a standing and/or sitting position. Sauna 200 includes seat 202, which may be a bench. In various embodiments, seat 202 includes a plurality of heaters, such as heaters 204. Moreover, walls of sauna 200, such as first wall 206 and second wall 208, may each include pluralities of heaters as well, such as heaters 210 and heaters 212. In various embodiments, heaters 204, heaters 210, and heaters 212 may all include the same type of heating element, or may include different types of heating elements. For example, the heating elements may be infrared heating elements configured to emit one or more of near infrared, mid infrared, or far infrared wavelengths. Accordingly, each of the heating elements may be configured to emit a specific infrared wavelength, such as just far infrared wavelengths, or the entire band of near infrared, mid infrared, and far infrared wavelengths. In one specific example, the heating elements may be carbon fiber impregnated heating elements.

In various embodiments, one or more components of sauna 200 are configured to reduce ELF radiation transmissions within sauna 200. More specifically, power cables coupled to the heaters may be configured to reduce such ELF radiation transmission. For example, power lines may be placed within conduit, and such conduit may be used to route the power lines to heating elements, such as heating elements of heaters 204, heaters 210, and heaters 212. The conduit may be made of a conductive material and electrically coupled to a circuit ground. For example, the conduit may be made of a conductive metal that is coupled to ground. In this way, the power lines may be sealed within a conductive and electrically grounded conduit.

As discussed above, sauna 200 may be configured to be coupled to halotherapy module 100 via one or more connections, as may be provided by a docking port, such as port 230. Accordingly, port 230 may be configured to provide mechanical coupling with halotherapy module 100 such that halotherapy module is housed within a recessed portion of port 230. Moreover, one or more data connections included in port 230 may provide communicative coupling between controller 220 and one or more components of halotherapy module 100, such as controller 116. In various embodiments, halotherapy module 100 may be configured such that controller 220 has direct access to components of halotherapy module 100, such as aerosolizer 104, and may control operation of such components by bypassing controller 116. In various embodiments, halotherapy module 100 may be controlled in combination with heaters 204, 210, and 212 to implement a comprehensive therapeutic program that combines the application of infrared heat and halotherapy. Accordingly, implementation of a sauna treatment program may be handled by controller 220 and a controller of halotherapy module 100, such as controller 116, such that the two controllers operate in coordination and in accordance with the sauna treatment program to control operation of the heaters and aerosolizer that may be included in sauna 200.

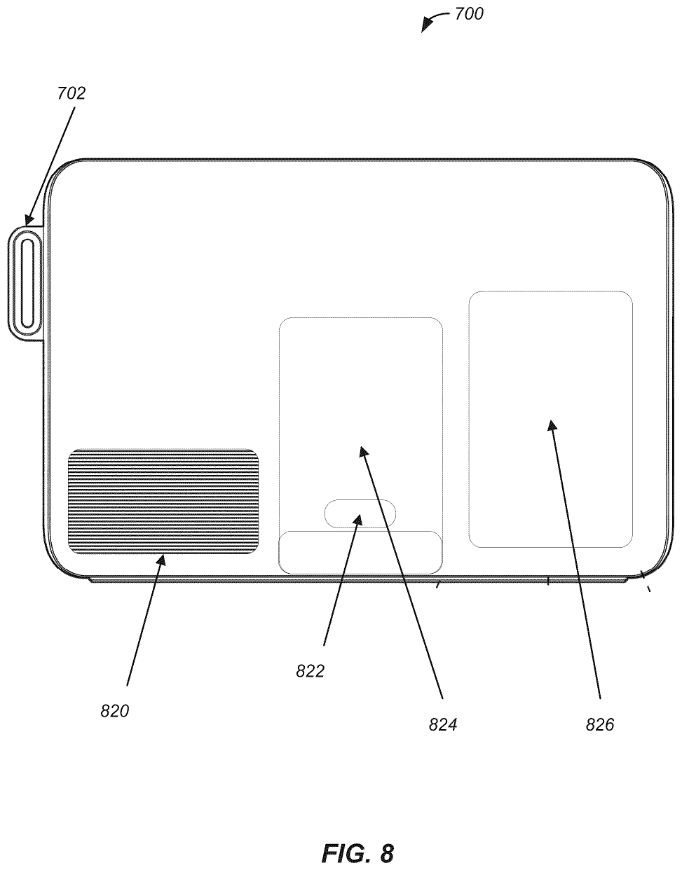

Sauna 200 may also include door 214 which may be coupled to the rest of the enclosure of sauna 200 via one or more couplers, such as coupler 216. In some embodiments, coupler 216 may be a hinge that is configured to provide free rotation of door 214. Sauna 200 may further include controller 220 which may include one or more processing components, as discussed in greater detail with reference to FIG. 8, which are configured to control the operation of heating elements, such as heaters 204, heaters 210, and heaters 212. As will be discussed in greater detail below, controller 220 may control the operation of the heaters independently and/or in groups.

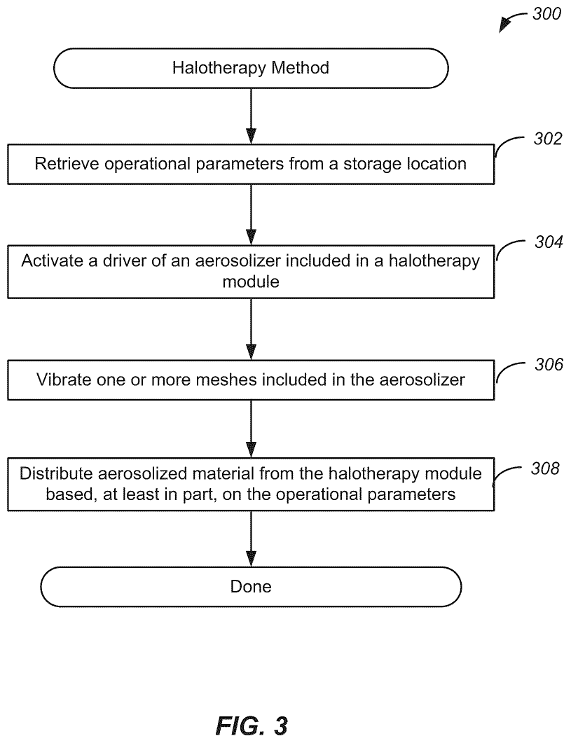

FIG. 3 illustrates a technique for using a halotherapy module, implemented in accordance with some embodiments. As will be discussed in greater detail below, a technique, such as technique 300, may be implemented to activate and utilized an aerosolizer in a configurable manner. As similarly discussed above, such activation of the aerosolizer may be used to implement one or more therapies, such as halotherapy.

Accordingly, technique 300 may commence with operation 302 during which operational parameters may be retrieved. As similarly discussed above, the operational parameters may determine a frequency and intensity of vibration of various meshes included in an aerosolizer. In various embodiments, the operational parameters may be stored in memory, or may be inferred based on a predetermined scheme, such as a look up table. In some embodiments, the operational parameters may be received from a user or an account associated with a user.

Technique 300 may proceed to operation 304 during which a driver may be activated. As similarly discussed above, the driver may be a mechanical driver configured to apply one or more axes of oscillatory motion to one or more meshes of the aerosolizer. Accordingly, the driver may be configured based on the received operational parameters to drive the meshes in accordance with a designated frequency and intensity.

Technique 300 may proceed to operation 306 during which the meshes may be vibrated. Accordingly, the driver may vibrate the meshes in accordance with the operational parameters discussed above. Moreover, a material may be fed from a reservoir or cartridge of the halotherapy module through the meshes while they are vibrated. In response to the material contacting the vibrating meshes, the material may form fine, uniform particles that are aerosolized.

Technique 300 may proceed to operation 308 during which the aerosolized material is distributed. Accordingly, in some embodiments, a fan may propel the aerosolized material from the aerosolizer into an ambient environment surrounding the halotherapy module. As will be discussed in greater detail below, the distribution of the aerosolized material may be modulated in accordance with additional operational parameters.

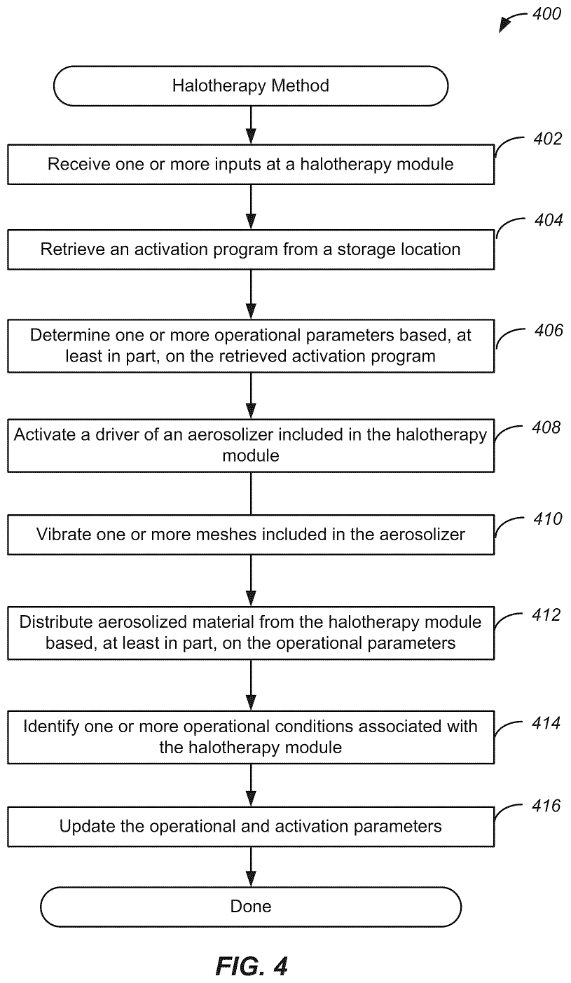

FIG. 4 illustrates a technique for using a halotherapy module, implemented in accordance with some embodiments. As similarly discussed above, a technique may be implemented to programmatically activate an aerosolizer in a configurable manner. As will be discussed in greater detail below, a technique, such as technique 400, may be implemented to enable programmatic activation of the aerosolizer may be used to implement one or more therapies, such as halotherapy.

Accordingly, technique 400 may commence with operation 402 during which one or more inputs may be received. In various embodiments, the input may be received form a user, and may be received via one or more input switches, an input device, or an external computing device coupled to the halotherapy module. In some embodiments, the external computing device may be a mobile device, and the halotherapy module may be in communication with an application executed on the mobile device. In various embodiments, the one or more inputs may identify one or more activation and operational parameters associated with the halotherapy module. For example, the input may identify the selection of a particular therapeutic program which may have been previously downloaded and stored in memory.

Technique 400 may proceed to operation 404 during which an activation program may be retrieved. In various embodiments, the activation program may identify a sequence of operational parameters associated with a selected therapy. As discussed above, the activation program may have been previously stored in memory. Accordingly, the appropriate activation program identifying such a sequence may be retrieved from a memory device, and based, at least in part, on the received one or more inputs.

Technique 400 may proceed to operation 406 during which one or more operational and activation parameters may be determined. Accordingly, the operational and activation parameters may be identified and retrieved from the activation program. Accordingly, the activation program may explicitly define such parameters for the operation of the halotherapy module. In some embodiments, such operational and activation parameters may also be inferred based on one or more other programs, such as programs associated with heaters discussed in greater detail below. Accordingly, a controller of the halotherapy module may be configured to map other parameters to operational and activation parameters for the halotherapy module based on predetermined logic, such as a look up table.

Technique 400 may proceed to operation 408 during which a driver may be activated. As similarly discussed above, the driver is a mechanical driver configured to apply one or more axes of oscillatory motion to one or more meshes of the aerosolizer. Accordingly, the driver may be configured based on the determined operational parameters, and they may be used to drive the meshes in accordance with a designated frequency and intensity.

Technique 400 may proceed to operation 410 during which the meshes may be vibrated. Accordingly, as similarly discussed above, the driver may vibrate the meshes in accordance with the operational parameters. Moreover, a material may be fed from a reservoir or cartridge of the halotherapy module through the meshes while they are vibrated. In response to the material contacting the vibrating meshes, the material may form fine, uniform particles that are aerosolized.

Technique 400 may proceed to operation 412 during which the aerosolized material is distributed. Accordingly, as similarly discussed above, a fan may propel the aerosolized material from the aerosolizer into an ambient environment surrounding the halotherapy module. As will be discussed in greater detail below, the distribution of the aerosolized material may be modulated in accordance with additional operational parameters.

Technique 400 may proceed to operation 414 during which one or more operational conditions may be identified. In various embodiments, such operational conditions may be conditions associated with particular aspects of the operation of the halotherapy module as well as a sauna that the halotherapy module might be implemented in. For example, the operational conditions may identify a passage of a designated amount of time, a change in ambient temperature and/or humidity, a change in temperature of the halotherapy module, an activation or deactivation of one or more other components of the sauna, activation of a motion sensor, and/or an opening or closing of a sauna door. In various embodiments, such operational conditions may be identified based on one or more sensors included in the sauna and/or halotherapy module. Moreover, such operational conditions may be identified based on components of the sauna and/or halotherapy module, such as a timer.

Technique 400 may proceed to operation 416 during which the operational and activation parameters may be updated. Accordingly, in response to identifying the operational conditions during operation 414, the operational and activation parameters may be updated during operation 416 to update the operation of the aerosolizer and the halotherapy module. For example, in response to identifying a particular change in temperature and/or humidity, an output of the aerosolizer may be modulated by adjusting the operation of the driver and/or operation of the fan. In this way, closed loop control may be implemented for the operation of the halotherapy module.

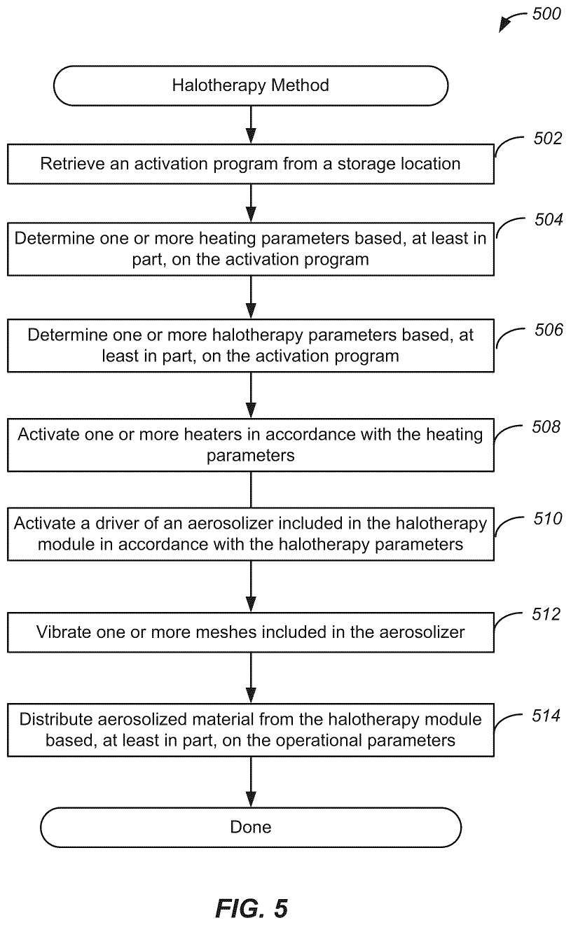

FIG. 5 illustrates a technique for using a halotherapy module, implemented in accordance with some embodiments. As similarly discussed above, a technique may be implemented to programmatically activate an aerosolizer in a configurable manner. As will be discussed in greater detail below, a technique, such as technique 500, may be implemented to enable programmatic activation of the aerosolizer in conjunction with other components of a sauna. Thus, the programmatic activation may be used to activate multiple different components of the sauna to provide a comprehensive therapy to the user.

Accordingly, technique 500 may commence with operation 502 during which an activation program may be retrieved. As similarly discussed above, the activation program may identify a sequence of operational parameters associated with a selected therapy. As also discussed above, the activation program may have been previously stored in memory. As will be discussed in greater detail below, the activation program may include activation parameters for multiple different components of a sauna. Accordingly, the appropriate activation program identifying such a sequence may be retrieved from a memory device, and based, at least in part, on the received one or more inputs.

Technique 500 may proceed to operation 504 during which one or more heating parameters may be determined. Accordingly, heating parameters may be identified based on the retrieved program, and may be used to identify a sequence of activation of different heaters included in the sauna, as well as an intensity and duration of activation for the heaters. As will be discussed in greater detail below, the heating parameters may identify different regions of activation that are configured based on a desired therapy.

Technique 500 may proceed to operation 506 during which halotherapy parameters may be determined. Accordingly, halotherapy parameters may be identified based on the retrieved program, and may be used to identify a sequence of activation of various components of the halotherapy module, such as a driver of an aerosolizer. As will be discussed in greater detail below, the halotherapy parameters may be implemented based, at least in part, on the heating parameters.

Technique 500 may proceed to operation 508 during which the heaters may be activated in accordance with the heating parameters. Accordingly, one or more of the heaters included in the sauna may be activated in accordance with the sequence of activation and intensity designated in the heating parameters. As similarly discussed above, the heaters may be activated in regions, and may be targeted to specific areas of a user's body based on a selected therapy. Moreover, the duration and intensity of activation may be customized based on a selected therapy. For example, a particular sequence of activation may be implemented for exercise recovery.

Technique 500 may proceed to operation 510 during which a driver may be activated. As similarly discussed above, the activation of the driver may be configured based on the determined halotherapy parameters, and may be used to drive the meshes in accordance with a designated frequency and intensity. In some embodiments, the activation of the driver may be configured based, at least in part, on the operation of other components of the sauna. For example, the activation may be timed to coincide with activation of the heaters.

Technique 500 may proceed to operation 512 during which the meshes may be vibrated. As similarly discussed above, the driver may vibrate the meshes in accordance with the halotherapy parameters. Moreover, a material may be fed from a reservoir or cartridge of the halotherapy module through the meshes while they are vibrated. In response to the material contacting the vibrating meshes, the material may form fine, uniform particles that are aerosolized.

Technique 500 may proceed to operation 514 during which the aerosolized material is distributed. Accordingly, in some embodiments, a fan may propel the aerosolized material from the aerosolizer into an ambient environment surrounding the halotherapy module. As discussed above, the ambient environment may be the enclosure of a sauna. Accordingly, during operation 514, the aerosolized material may be distributed throughout the interior of the sauna, thus providing halotherapy to the user that is using the sauna.

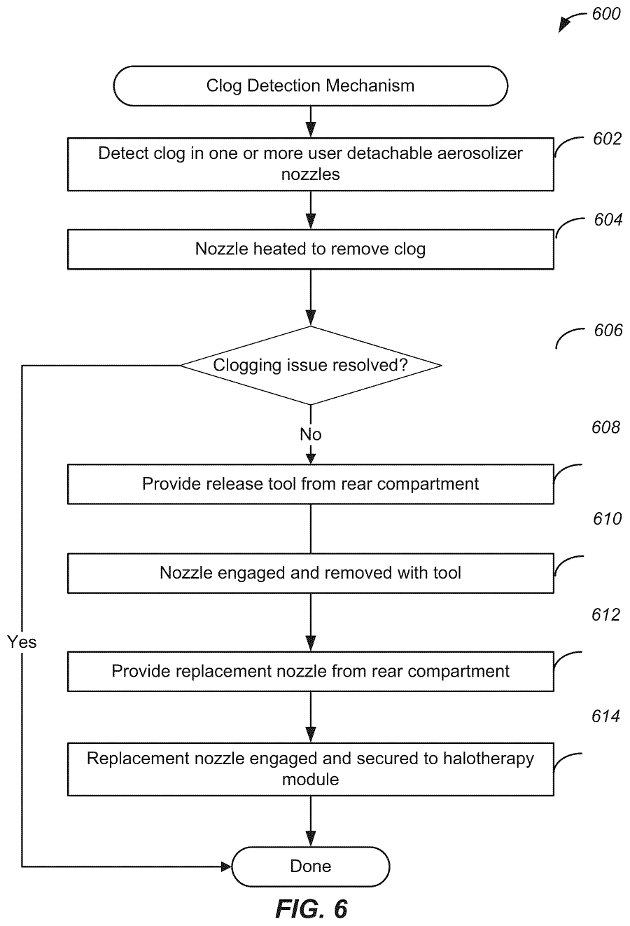

FIG. 6 illustrates one example of a clog detection mechanism. At 602, a clog is detected in one or more user detachable aerosolizer nozzles. A clog may be detected if no aerosolized salt particles are emitted through a particular nozzle, or if pressure builds in the aerosolizer unit. Acve, at 604 the user detachable aerosolizer nozzle is heated to potentially dissolve the clog. At 606, it is determined if the clogging issue is resolved. If the issue is resolved, the process is complete. If the issue is not yet resolved, a release tool is provided from a rear compartment of the aerosolizer or a halotherapy unit itself at 608. At 610, the nozzle is engaged with the tool and removed. Ipee, the nozzle is removed with a twisting motion of the tool, but other disengagement mechanisms are possible. At 612, a replacement nozzle is provided from a rear compartment. At 614, the replacement nozzle is engaged and secured with the tool onto the aerosolizer or halotherapy module.

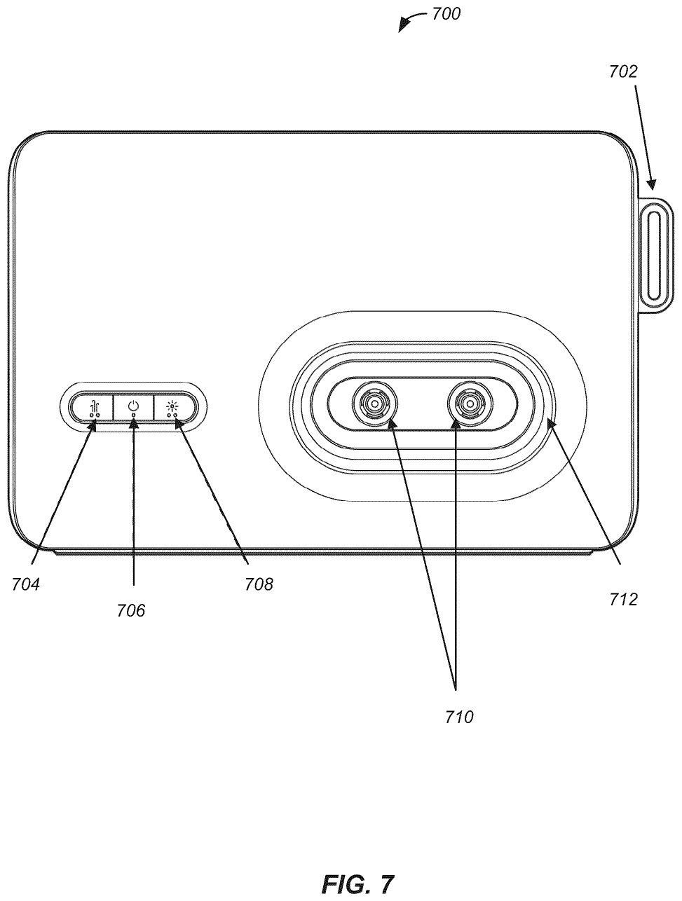

FIG. 7 illustrates an example of a front side of a halotherapy unit or aerosolizer 700 having a user replaceable aerosolizer nozzle. Acve, the aerosolizer includes a salt cartridge input unit 702. Ipee, the salt cartridge input unit 702 is a saline capsule holder. The aerosolizer 700 may also include a power button 706, a fan switch 704, and an emotive light control button 708. Emotive lights may be displayed in strip 712 and the aerosolizer emits a salt spray or aerosolilzed salt through user replaceable nozzles 710. The device may also include a heating element to potentially reduce the likelihood of clogged nozzles 710 or remove clogs in nozzles 710. It should be noted that a variety of configurations are possible.

FIG. 8 illustrates one example of a back side of a halotherapy unit or aerosolizer 700. The salt cartridge input unit 702 is seen at 702. The rear may also include an air intake 820 along with a mounting area 824 for mounting onto a sauna or onto the interior wall of a sauna. The aerosolizer 700 may also include a plug for a power cable 822 or may be battery powered. Ipee, compartment 826 includes a tool and replaceable nozzles for the aerosolizer.

FIG. 8 illustrates one example of a controller, configured in accordance with some embodiments. According to particular embodiments, a system 900 suitable for implementing particular embodiments of the present invention includes a processor 901, a memory 903, an interface 911, and a bus 916 (e.g., a PCI bus or other interconnection fabric) and operates as a controller, such as controller 220, configured to control the operation of heaters and/or a halotherapy module, as discussed above. When acting under the control of appropriate software or firmware, the processor 901 is responsible for receiving inputs and controlling the operation of heaters and heating elements and/or halotherapy module. In some embodiments, system 900 may be integrated in controller 220 discussed above, or may be implemented separately as a stand-alone component that provides functionalities for controller 220. Various specially configured devices can also be used in place of a processor 901 or in addition to processor 901. The interface 911 is typically configured to send and receive data. In some embodiments, such an interface may be configured to send packets or data segments over a network.

Particular examples of interfaces supported include Ethernet interfaces, cable interfaces, DSL interfaces, token ring interfaces, and the like. In addition, various very high-speed interfaces may be provided such as fast Ethernet interfaces, Gigabit Ethernet interfaces, ATM interfaces, HSSI interfaces, POS interfaces, FDDI interfaces and the like. Generally, these interfaces may include ports appropriate for communication with the appropriate system components. In some cases, they may also include an independent processor and, in some instances, volatile RAM. The independent processors may control communications-intensive tasks.

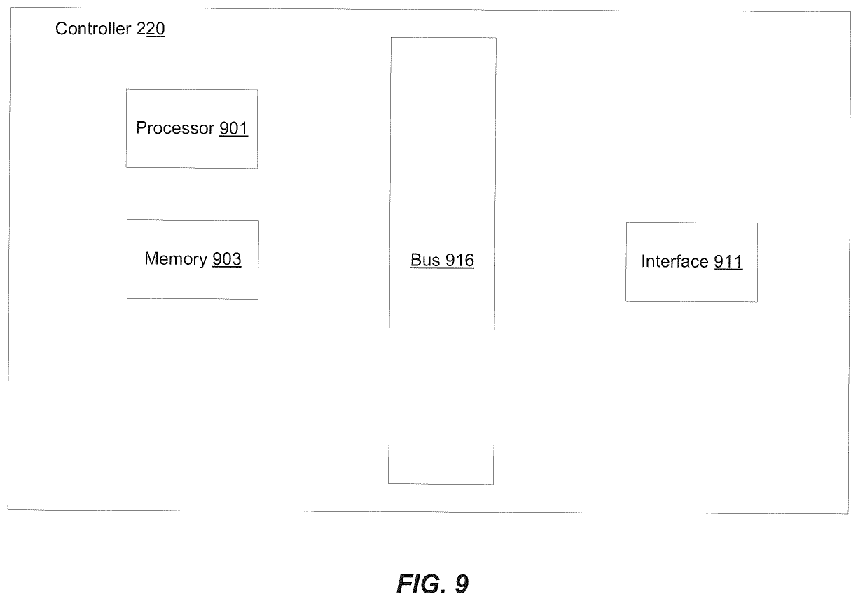

According to various embodiments, the system 900 is a controller configured to control the operation of heating elements in accordance with the modes and operations discussed above. For example, the system 900 may be configured to control heating elements as shown in FIG. 1. The controller may include one or more hardware elements as shown in FIG. 9. In some implementations, one or more of the controller components may be virtualized. For example, a physical server may be configured in a localized or cloud environment. Although a particular controller is described, it should be recognized that a variety of alternative configurations are possible. For example, the modules may be implemented on another device connected to a server.

In the foregoing specification, the invention has been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

What is claimed is:

1. A system comprising:
a receiving port configured to receive a saline solution, wherein the saline solution comprises saline solution particles having diameters between 2 μm and 12 μm;
a sensor configured to generate one or more measurements based on ambient conditions of a sauna;
an aerosolizer configured to aerosolize the saline solution in response to receiving a signal, wherein the aerosol comprises dry salt particles having diameters between .5020 μm and 3.0122 μm;
a user detachable aerosolizer nozzle, wherein the user detachable aerosolizer nozzle is detached using a tool included with the aerosolizer;
a controller comprising one or more processors configured to generate the signal provided to the aerosolizer, and further configured to control operation of the aerosolizer via the signal.

2. The system of claim 1, wherein the aerosolizer comprises:
a driver; and
a plurality of meshes.

3. The system of claim 2, wherein the driver is a mechanical driver configured to vibrate at least one of the plurality of meshes.

4. The system of claim 3, wherein the vibration of the at least one of the plurality of meshes is implemented based, at least in part, on the signal.

5. The system of claim 2, wherein the plurality of meshes comprises a plurality of layers each having a different dimension and geometry.

6. The system of claim 1, wherein the material is a saline solution.

7. The system of claim 1, wherein the housing is configured to be removably coupled to an interior of a sauna.

8. A device comprising:
a housing;
an aerosolizer configured to aerosolize a material in response to receiving a signal, wherein the material is a saline solution having a salt concentration of approximately 3% and a saline solution particle diameter between 2 μm and 12 μm; and
a controller comprising one or more processors configured to generate the signal provided to the aerosolizer, and further configured to control operation of the aerosolizer via the signal, wherein the aerosol includes resultant dry salt particle having diameters between .5020 μm and 3.0122 μm.

9. The device of claim 8, wherein the aerosolizer comprises:
a driver; and
a plurality of meshes.

10. The device of claim 9, wherein the driver is a mechanical driver configured to vibrate at least one of the plurality of meshes.

11. The device of claim 10, wherein the vibration of the at least one of the plurality of meshes is implemented based, at least in part, on the signal.

12. The device of claim 9, wherein the plurality of meshes comprises a plurality of layers each having a different dimension and geometry.

13. The device of claim 8, wherein the material is a saline solution.

14. The device of claim 8, wherein the housing is configured to be removably coupled to an interior of a sauna.

15. A method comprising:
receiving, at a port, a cartridge configured to store a material capable of being aerosolized, wherein the material is a saline solution having a 2%-5% saline solution concentration;
generating, using a sensor, one or more measurements based on ambient conditions of a housing coupled to the port;
generating, using a controller, a signal based on a plurality of operational parameters, wherein the signal is a control signal for an aerosolizer included in the housing; and
aerosolizing contents of the cartridge in response to receiving a signal, wherein the aerosol includes resultant dry salt particles having a diameter substantially between .5 μm and 3 μm, wherein the aerosol passes through a user detachable aerosolizer nozzle.

16. The method of claim 15, wherein the aerosolizing comprises:
vibrating a plurality of meshes.

17. The method of claim 16, wherein the vibrating comprises:
using a mechanical driver to vibrate at least one of the plurality of meshes.

18. The method of claim 17, wherein the vibration of the at least one of the plurality of meshes is implemented based, at least in part, on the signal.

19. The method of claim 16, wherein the plurality of meshes comprises a plurality of layers each having a different dimension and geometry.

20. The method of claim 15, wherein the aerosolizer nozzle is detached using a tool included with the aerosolizer.
